(19) United States
(12) Patent Application Publication
(10) Pub. No.: US 2016/0169790 A1
(43) Pub. Date: Jun. 16, 2016

(54) SYSTEM AND METHOD FOR TESTING EXPOSURE OF A HYDROCARBON ON A COMPONENT

(71) Applicant: SHELL OIL COMPANY, Houston, TX (US)

(72) Inventors: Hanane Belmokaddem BENNIS, Richmond, TX (US); Timothy Michael SHEA, The Woodlands, TX (US); Michael Lane BAKER, Richmond, TX (US)

(21) Appl. No.: 14/963,284

(22) Filed: Dec. 9, 2015

Related U.S. Application Data
(60) Provisional application No. 62/090,597, filed on Dec. 11, 2014.

(51) Int. Cl. G01N 17/00; G01N 33/28; G01N 11/00

(52) U.S. Cl. CPC G01N 17/006; G01N 11/00; G01N 33/28

(57) ABSTRACT

A tester, systems and method for testing hydrocarbons are provided. The tester includes a test module supporting the component thereabout. The test module is operatively connectable to a fluid source to receive the hydrocarbon therefrom. A recirculation circuit is operatively connectable to the fluid circuit about the test module to recirculate the hydrocarbon thereabout whereby the hydrocarbon is passed over at least a portion of the component. The method involves passing the hydrocarbon over at least a portion of a plurality of the components, recirculating the hydrocarbon over the portion of each of the components, and performing a material test on each of the components.

The present application claims the benefit of pending U.S. Provisional Application Ser. No. 62/090,597, filed 11 Dec. 2014, the entire disclosure of which is hereby incorporated by reference.

### FIELD OF THE INVENTION

The present disclosure relates generally to hydrocarbons. More specifically, the present disclosure relates to techniques for testing hydrocarbons, such as fuels, additives, and/or lubricants.

### BACKGROUND OF THE INVENTION

Hydrocarbons, such as fuel, may be used in machinery, such as engines, to provide power. For example, hydrocarbons may be housed in a fuel tank and passed via fuel lines to engines, and ignited to generate power. The composition of the fuel may affect the function of the equipment used with the fuel. In some cases, the hydrocarbons may damage equipment, such as the fuel tanks and fuel lines that contact the hydrocarbons during operation.

Tests have been developed for evaluating fuels. Examples of tests involving fuels are provided in U.S. Pat. No. 6,125,690 and JP2009264394. Standards have also been provided for evaluating fuels. For example, the American Society for Testing and Materials (ASTM) International has developed ASTM Designation: D7826-13 (available at www.astm.org) for evaluating gasoline.

### SUMMARY OF THE INVENTION

In at least one aspect, the disclosure relates to a tester for testing exposure of a hydrocarbon on a component. The hydrocarbon is stored in a fluid source. The tester includes a test module supporting the component thereabout and a recirculation circuit. The test module is operatively connectable to the fluid source to receive the hydrocarbon therefrom. The recirculation circuit operatively connectable about the test module to recirculate the hydrocarbon thereabout whereby the hydrocarbon is passed over at least a portion of the component.

In another aspect, the disclosure relates to a system for testing exposure of a hydrocarbon on a component. The system includes at least one fluid source having the hydrocarbon therein, a fluid circuit in fluid communication with the fluid source, and a plurality of testers operatively connectable to the fluid circuit to receive the hydrocarbon therefrom. Each of the testers includes a test module supporting the component thereabout and a plurality of recirculation circuits. Each of the recirculation circuits is operatively connectable to the fluid circuit about a corresponding one of the plurality of test modules to recirculate the hydrocarbon thereabout whereby the hydrocarbon is passed over at least a portion of the component.

Finally, in another aspect, the disclosure relates to a method for testing exposure of a hydrocarbon on a component. The method involves passing the hydrocarbon over at least a portion of each of a plurality of the components, recirculating the hydrocarbon over the portion of each of the components, and performing a material test on each of the components.

### BRIEF DESCRIPTION OF THE DRAWINGS

So that the above recited features and advantages of the disclosure may be understood in detail, a more particular description of the disclosure, briefly summarized above, may be had by reference to the embodiments thereof that are illustrated in the appended drawings. It is to be noted, however, that the appended drawings illustrate only typical embodiments of this disclosure and are, therefore, not to be considered limiting of its scope. The figures are not necessarily to scale, and certain features and certain views of the figures may be shown exaggerated in scale or in schematic in the interest of clarity and conciseness.

FIG. 1 is a schematic diagram depicting a system with testers having test modules for testing exposure of a hydrocarbon on a component, in accordance with the present disclosure;

FIGS. 2A-2B are schematic diagrams depicting an immersion test module usable in the system of FIG. 1, in accordance with the present disclosure;

FIGS. 3A-3B are schematic diagrams depicting a partial-exposure test module usable in the system of FIG. 1, in accordance with the present disclosure;

FIG. 4 is a schematic diagram depicting a flow-through test module usable in the system of FIG. 1, in accordance with the present disclosure; and

FIGS. 5A and 5B are flow charts depicting methods for testing exposure of a hydrocarbon on a component, in accordance with the present disclosure.

### DETAILED DESCRIPTION OF THE INVENTION

The description that follows includes exemplary apparatuses, methods, techniques, and instruction sequences that embody techniques of the inventive subject matter. However, it is understood that the described embodiments may be practiced without these specific details.

The disclosure relates to techniques for testing exposure of hydrocarbons, such as fuels and additives, on components, such as coupons, gaskets, tubes, or other materials. The tests may be performed using a modular test system with one or more testers. Each tester includes a test module to support the component, and a recirculation circuit to recirculate the hydrocarbon about the test module. The hydrocarbon may be passed via a fluid circuit about one or more testers. Sensors and/or visual detectors may be provided to sense changes in fluid parameters (e.g., viscosity, temperature, flow rate, etc.) of the hydrocarbon, material parameters (e.g., color, material properties, etc.) of the components, and/or other portions of the system. Material tests (e.g., hardness, tensile, etc.) may also be performed on the components.

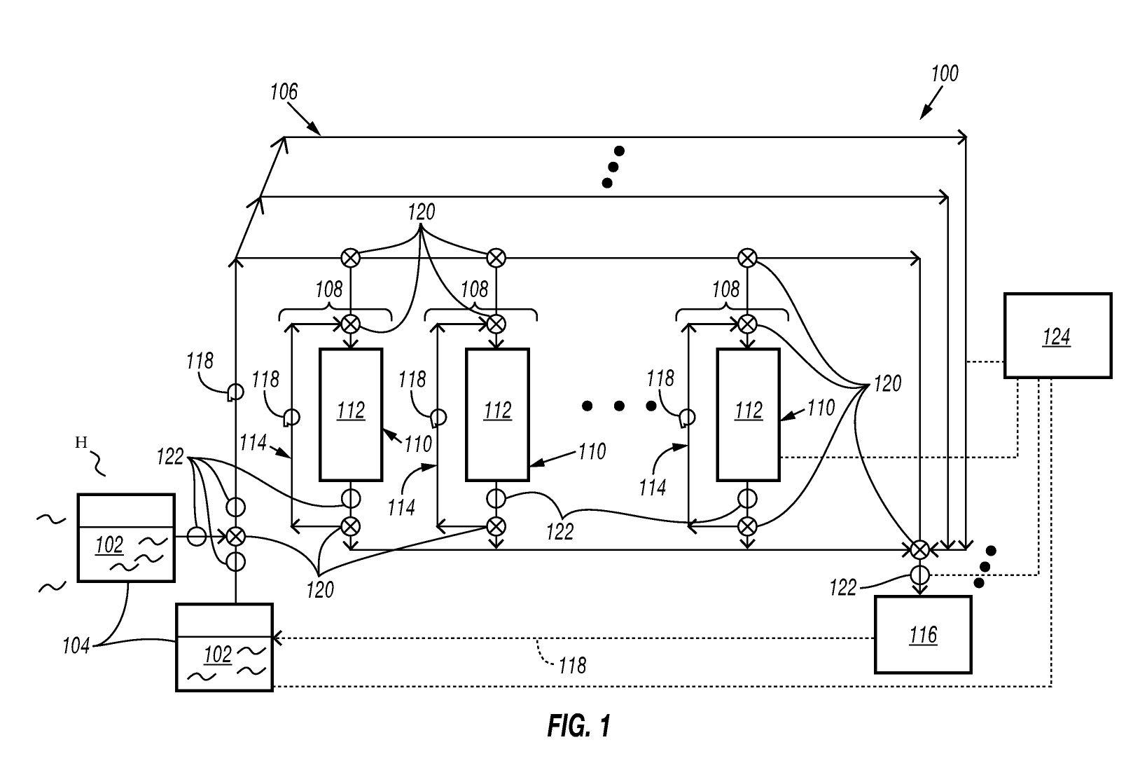

FIG. 1 illustrates a test system 100 usable for evaluating a hydrocarbon 102. The test system 100 includes fluid sources 104, a fluid circuit 106, and testers 108. One or more fluid sources 104 may be provided to store the hydrocarbon 102. The fluid sources 104 may have the same or different hydrocarbons.

While the test system 100 is described with respect to exposure of a hydrocarbon to a component, the test system 100 may be used for testing exposure of any fluid to any item. The hydrocarbon may be, for example, a fuel, such as gasoline, diesel, and/or additives usable therewith. Other fuels may be envisioned, such as ethanol, biodiesel, propane, compressed natural gas, battery, hydrogen or fluid. An example hydrocarbon usable with the test system would be a fuel having a viscosity of from about 0.5 mm²/sec to about 1,500 mm²/s or more, and a temperature of about 40 degrees C.

The fluid sources 104 are fluidly coupled to the fluid circuit 106. The fluid circuit 106 includes flowlines fluidly coupling the fluid source(s) 104 to one or more of the testers 108. The flowlines may be fluid lines made of materials, such as tygon tubing, stainless steel, etc. As also shown by H in FIG. 1, the fluid sources 104 (and/or other portions of the fluid circuit 106) may be heated. Heat may be applied to the hydrocarbon 102 before or after passing through the fluid circuit 106.

The testers 108 include test modules 110 for supporting the components 112 for testing. The test modules 110 may be positioned in fluid communication with the fluid circuit 106 to receive the hydrocarbon 102 therefrom and to expose at least a portion of the component 112 thereto as schematically shown.

A recirculation circuit 114 is provided about each of the test modules 110 to recirculate the hydrocarbon 102 therethrough as indicated by the arrows. The recirculation circuit 114 may be coupled to the test module 110 and/or the fluid circuit 106 to repeatedly pass the hydrocarbon 102 through the test module 110. Once complete the hydrocarbon 102 may be discharged from the tester 108.

A variety of flowlines of the fluid circuit 106 may be configured to accommodate a variety of configurations of the system 100. The flowlines as shown couples the fluid sources 104 to multiple test modules 108 that may be tested simultaneously, in pattern (e.g., series), or independently. On or more sets, rows, columns, or other patterns of test modules 108 may be provided. As indicated by the ellipses ( . . . ), the system 100 may be expandable to accommodate any combination of components of the system as provided herein.

The system 100 may be modular to provide removable components 112 and/or reconfigurable to accommodate a variety of arrangements. One or more test modules 110 may be provided about the system 100 to permit testing of one or more components and/or hyrdocarbons at the same time for comparison. In some cases, a test cycle of various components and/or hydrocarbons may be varied to provide desired experiments designed to determine various characteristics.

The flowlines of the fluid circuit 106 may also be configured to provide for discharge of the fluid from the testers 108 to a waste bin 116. As indicated by dashed line 118, a recirculation flowline may be provided to pass hydrocarbon 102 from waste bin 116 back to the fluid source 104 and/or the fluid circuit 106 for reuse. The hydrocarbon 102 may be passed to one or more waste bins 116 and/or recirculated to one of more of the testers 108 as desired.

The fluid circuit 106 may be provided with a variety of flow control devices, such as pumps 118, valves 120, etc. Pump 118 in fluid circuit 106 may be used to pump the hydrocarbon 102 through the flow circuit 106. Pump 118 in recirculation circuit 114 may be used to pump the hydrocarbon 102 through the test module 108. Valves 120 may be positioned at various locations along the fluid circuit 106 to direct hydrocarbon 102 to desired locations. For example, on completion of a test, one or more testers 108 may be fluidly isolated by valves and/or be removable from the fluid circuit 106.

The fluid circuit 106 provides a closed system usable with a variety of fluid, including flammable, toxic, evaporable, or other sensitive fluids. For example, the fluid may be an aviation gasoline prone to evaporation which may affect the results (e.g., where evaporation of the aviation gas may cause the percent of heavier components in the fluid blend higher).

The system 100 may also be provided with sensors 122. The sensors 122 may be, for example, gauges, monitors, electrodes, or other sensors capable of measuring a variety of system (e.g., flow rate), fluid (e.g., temperature, viscosity, composition, etc.) and/or material (e.g., material, temperature) parameters. The sensors 122 may be positioned, for example, along the flowlines of the fluid circuit 106 and/or the recirculation circuit 114. The sensors 112 may be coupled to a central unit 124 for collection and/or analysis.

The central unit 124 may be provided with a variety of communication, power, storage, control, and/or analysis functions. For example, the central unit 124 may include a processor (e.g., central processing unit (CPU)), database, transceiver, controller, and/or other devices. The central unit 124 may be located at, or a distance from, the fluid circuit 106 and in communication therewith. One or more central units 124 at various locations may be provided. The central units 124 may be used, for example, to collect, compare, and report on various data collected involving one or more testers, hydrocarbons, and/or components.

The test system 100 may be operated automatically using central unit 124 and/or manually. The hydrocarbon 102 may be passed through the fluid circuit 106 to the test modules 110. The recirculation loop 114 may be used to recirculate the same hydrocarbon through the test module(s) 110 one or more times. The hydrocarbon 102 may continue to pass through the test module 110 one or more times until a test is completed.

The test may be completed based on a predetermined test time or upon detection of a change in the system 100. For example, the sensors 122 may detect a change in material and/or fluid parameters indicating the test should be terminated. In some cases, visual inspection of the component 112 may indicate that a color change has occurred. Once completed, the test may be automatically or manually terminated.

Once terminated, the test module 110 may be fluidly isolated from the fluid circuit 106 by valves 120 and the hydrocarbon 102 may be allowed to drain from the test module to waste bin 116. The component 112 may be visible through the test module 110, or removed from the test module 110 for inspection and/or further testing. Further testing may include, for example, material testing, such as hardness, tensile, compressive, or other known material tests. Data from these tests may be provided to the central unit 124 for further analysis alone or with other data, such as data collected during the test by system 100. The data may be stored, distributed, analyzed, reported, etc.

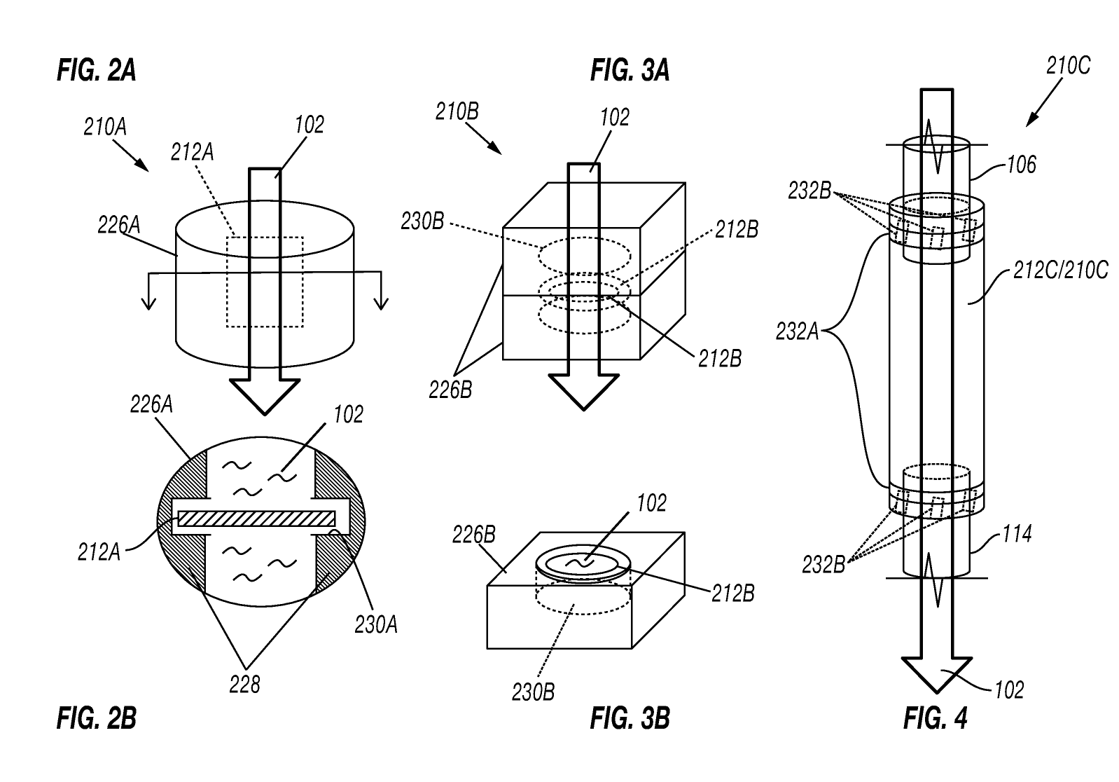

FIGS. 2A-4 show various example test modules 210a-c that may be used with various components 212a-c. FIGS. 2A-2B depict an example immersion test module 210a. FIGS. 3A-3C depict an example partial-exposure test module 210b. FIG. 4 depicts an example flow-through test module 210c. Each example test module 210a-c supports the component 212a-c, respectively, therein such that the hydrocarbon 102 passes along a desired portion of the component.

In the example of FIGS. 2A2B, the component 212a is a coupon (e.g., metal plate) immersed within the test module 210a. The test module 210a includes a housing 226a with supports 228 therein. The housing 226a as shown is elliptical in shape, but could be in any form. The supports 228 are depicted as defining an immersion receptacle 230a for receiving the coupon 212a. As shown, the housing 226a loosely receives the coupon 212a such that most if not all portions of the coupon 212a are exposable to (e.g., immersed in) the hydrocarbon 102 passing through the housing 226a as indicated by the arrow. In this configuration, the coupon 212a may be immersed within the hydrocarbon 102.

In the example of FIGS. 3A-3B, the component 212b is an elliptical gasket insertable into the test module 210b. The test module 210b includes a two-portion housing 226b. As shown, the housing 226b is rectangular with two connectable portions, with each portion having an interference fit receptacle 230b shaped to snugly receive an outer surface of the gasket 212b. The housing portions are matable to fittingly receive the gasket 212b therein such that fluid passing through the housing 226b passes along an inner surface of the gasket 212b, but cannot engage an outer surface of the gasket 212b. Thus, the outer surface of the gasket 212b is in contact with a surface of the housing 226b along the receptacle 230b to prevent hydrocarbon contact therealong, and only the inner surface of the gasket 212b may be exposed.

While FIGS. 3A and 3B show a rectangular housing 226b in two portions, the housing can be any shape with one or more housing portions to receive and/or support the gasket 212b therein. The receptacle 230b may be shaped to receive the component for partial or full exposure to the hydrocarbon 102. The receptacle 230b may be larger than the component or a smaller component may be placed in a larger receptacle 230b to permit full immersion when desired.

In the example of FIG. 4, the component 212c is a tube connectable to a flowline of the flow circuit 106 and the recirculation circuit 114. In this case, the tube 212c is its own self-contained module 210c. The tube 212c is connected by connectors 232a, b to the flowlines of the flow circuits 106 and/or recirculation loop 114. In this configuration, the tube 212c is connected so that hydrocarbon 102 may pass through the flow circuit 106, through tube 212c, and out the recirculation loop 114 such that an inner surface of the tube 212c is exposed to the hydrocarbon 102.

As shown in FIG. 4, the connectors 232a are clamps forming a band tightened around the tube 212c to secure the tube 212c to the flowlines. As also shown, the connectors 232b are barbs 232b also connecting the tube 212c to the flowlines. The barbs 232b may be on the tube 212c and/or the flowlines. The barbs 232b may have points to grip the tube 212c and secure the tube 212c to the flowline. One or more of a variety of connectors, such as clamps and/or barbs, may be used to connect the components/test modules 212c/210a about the flow circuit 106 and/or recirculation circuit 114.

While FIGS. 2A-2C show example test modules that may be used in the system 100 and/or with various components, it will be appreciated that a variety of test modules may be used to support one or more components for exposure to hydrocarbon. The test modules may be configured to permit exposure of part, or all, of the component to the hydrocarbon.

The components may be any component that may be exposed to the hydrocarbon, such as samples (e.g., coupons) of material used for fuel tanks, seals (or gaskets), fuel lines, and motor materials. The test modules may be made of any material, such as glass, stainless steel, aluminum, etc.) For example, the test modules may optionally be clear or have a window to permit visual inspection of the components during testing. Visual inspection may be used to monitor changes and may indicate completion of a test. Various combinations of the test modules may also be provided.

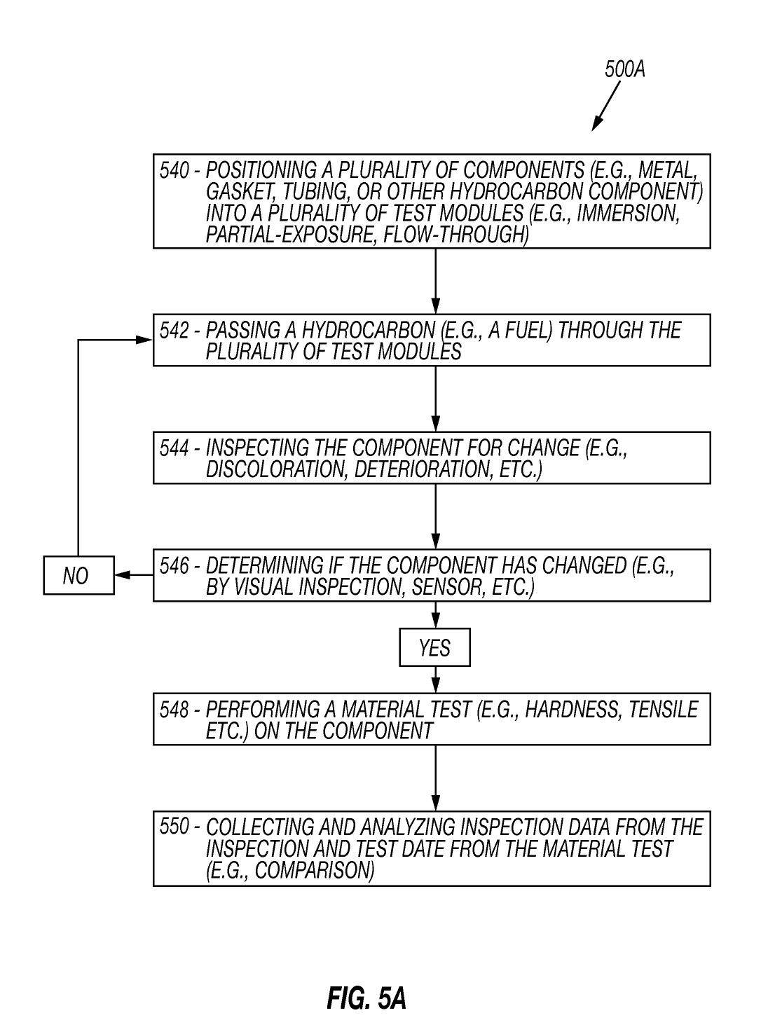

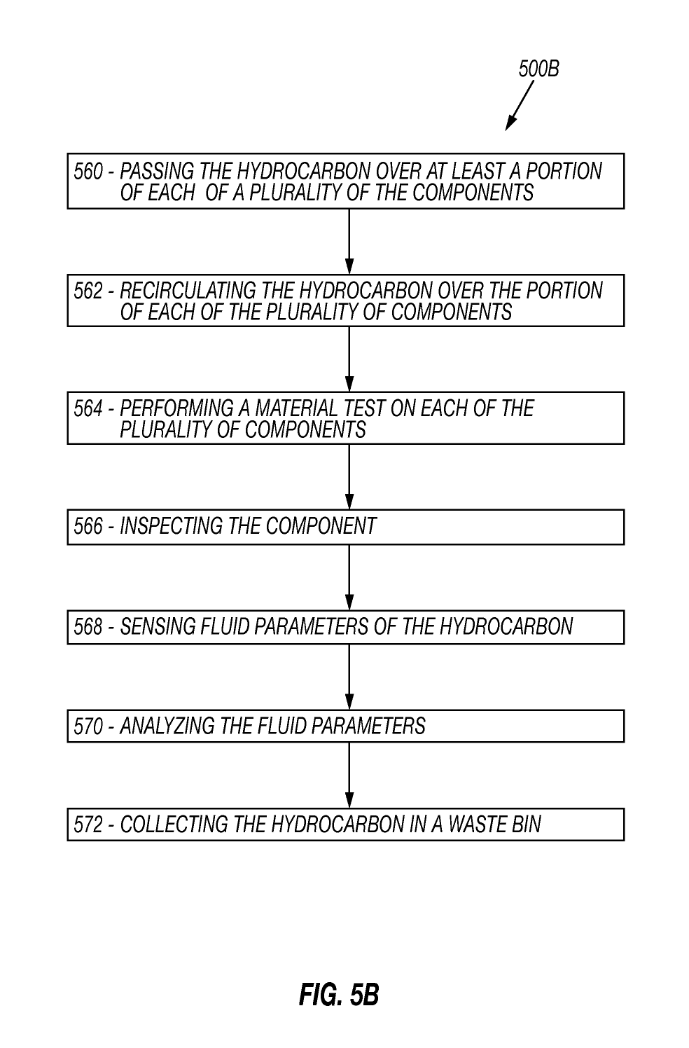

FIGS. 5A and 5B depict methods 500a,b of testing exposure of a hydrocarbon on a component, such as the hydrocarbon and components shown herein. The method 500a may involve 540—positioning a plurality of components (e.g., metal, gasket, tubing, or other hydrocarbon component) into a plurality of test modules (e.g., immersion, partial-exposure, and flow-through). The method may also involve 542—passing a hydrocarbon (e.g., a fuel) through the plurality of test modules, 544—inspecting (e.g., visually inspecting, removing from the component for inspection, inspecting with sensor) the component for change (e.g., discoloration, deterioration, etc.), and 546—determining if the component has changed (e.g., by visual inspection, sensor, etc.) The passing 542 may be performed one or more times. In some cases 544-546 may be skipped and the passing 542 may be performed for a given time frame before proceeding to 548-550.

If the component has not changed, 542-546 may continue until a change is detected. If the component has changed, the method may continue with 548—performing a material test (e.g., hardness, tensile, etc.) on the component, and 550—collecting and analyzing inspection data from the inspection and test date from the material test (e.g., comparison).

The method 500b involves 560—passing the hydrocarbon over at least a portion of each of a plurality of the components, 562—recirculating the hydrocarbon over the portion of each of the plurality of components, and 564—performing a material test on each of the plurality of components.

The method may also involve 566—inspecting the component (e.g., by visual or sensors), 568—sensing fluid parameters of the hydrocarbon (e.g., by sensors 122), 570—analyzing the fluid parameters (e.g., by central unit 124), and/or 572—collecting the hydrocarbon in a waste bin (e.g., 116). The analyzing may involve comparing each of the plurality of components. The recirculating may involve recirculating the hydrocarbon about each one of the plurality of components (e.g., by 114) and/or recirculating the hydrocarbon about all of the plurality of components (e.g., by 118).

The method may be repeated as desired and performed in any order.

While the embodiments are described with reference to various implementations and exploitations, it will be understood that these embodiments are illustrative and that the scope of the inventive subject matter is not limited to them. Many variations, modifications, additions and improvements are possible. For example, one or more testers and/or test modules with one or more test components and/or hydrocarbons tested by visual, sensor, and/or material testing over time as described herein. While the invention is illustrated making reference to one or more a specific combinations of features and measures, many of those features and measures are functionally independent from other features and measures such that they can be equally or similarly applied independently in other embodiments or combinations.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

That which is claimed is:

1. A tester for testing exposure of a hydrocarbon on a component, the hydrocarbon stored in a fluid source, the tester comprising:
a test module supporting the component thereabout, the test module operatively connectable to the fluid source to receive the hydrocarbon therefrom; and
a recirculation circuit operatively connectable about the test module to recirculate the hydrocarbon thereabout whereby the hydrocarbon is passed over at least a portion of the component.

2. The tester of claim 1, wherein the test module comprises an immersion housing having a component support to supportingly receive the component.

3. The tester of claim 1, wherein the test module comprises a partial-exposure housing having a component cavity shaped to fittingly receive an outer surface of the component.

4. The tester of claim 1, wherein the test module comprises flow-through connectors operatively connecting the component to the fluid source and the recirculation circuit.

5. The tester of claim 4, wherein the flow-through connector comprises at least one of a clamp and a barb.

6. The tester of claim 1, wherein the recirculation circuit comprises a flowline operatively connectable to an upstream end and a downstream end of the test module to recirculate the hydrocarbon thereabout.

7. The tester of claim 1, wherein the hydrocarbon is a fuel having a viscosity of at least 0.5 mm²/sec at 40 degrees C.

8. The tester of claim 1, wherein the component is at least one of a coupon, a gasket, a tube, and combinations thereof.

9. The tester of claim 1, wherein the test module is at least partially clear to enable visual inspection of the component and wherein the test module comprises at least one of glass, stainless steel, and aluminum.

10. A system for testing exposure of a hydrocarbon on a component, the system comprising:
at least one fluid source having the hydrocarbon therein;
a fluid circuit in fluid communication with the fluid source; and
a plurality of testers operatively connectable to the fluid circuit to receive the hydrocarbon therefrom, each of the plurality of testers comprising:
a test module supporting the component thereabout; and
a plurality of recirculation circuits, each of the plurality of recirculation circuits operatively connectable to the fluid circuit about a corresponding one of the plurality of test modules to recirculate the hydrocarbon thereabout whereby the hydrocarbon is passed over at least a portion of the component.

11. The system of claim 10, further comprising at least one waste unit operatively connectable to the fluid circuit.

12. The system of claim 10, further comprising at least one sensor operatively connectable about at least one of the at least one fluid source, the fluid circuit, the test modules, and the recirculation circuits.

13. The system of claim 10, further comprising at least one of a pump, a valve, and a flowline.

14. The system of claim 10, further comprising an analyzer operatively connectable to at least one of the at least one fluid source, the fluid circuit, the test modules, and the recirculation circuits.

15. A method for testing exposure of a hydrocarbon on a component, the method comprising:
a) passing the hydrocarbon over at least a portion of each of a plurality of the components;
b) recirculating the hydrocarbon over the portion of each of the plurality of the components; and
c) performing a material test on each of the plurality of components.

16. The method of claim 15, further comprising visually inspecting the component.

17. The method of claim 16, wherein the inspecting comprises monitoring changes to the components.

18. The method of claim 15, further comprising sensing fluid parameters of the hydrocarbon.

19. The method of claim 15, further comprising analyzing the fluid parameters, the analyzing comprising comparing each of the plurality of components.

20. The method of claim 15, further comprising collecting the hydrocarbon in a waste bin after the recirculating.

21. The method of claim 20, wherein the recirculating comprises at least one of recirculating the hydrocarbon about each one of the plurality of components and recirculating the hydrocarbon about all of the plurality of components.
